(19) United States
(12) Patent Application Publication
(10) Pub. No.: US 2023/0190987 A1
(43) Pub. Date: Jun. 22, 2023

(54) Snag Resistant Blood Absorbence Pad And Method For Use By A Patient Having Braces

(71) Applicant: Amy Silva, Sterling Heights, MI (US)

(72) Inventor: Amy Silva, Sterling Heights, MI (US)

(21) Appl. No.: 17/730,590

(22) Filed: Apr. 27, 2022

Related U.S. Application Data
(60) Provisional application No. 63/290,888, filed on Dec. 17, 2021.

(51) Int. Cl. A61L 15/42

(52) U.S. Cl. CPC A61L 15/42; A61F 2013/00217

(57) ABSTRACT

An arrangement and pad for absorbing blood by one or more gauze strips positioned over a socket formed by removal of a tooth. The gauze is enclosed within an enclosure defined between two pieces of a smooth fabric material such as tightly woven smooth cotton which greatly reduces any tendency to snag the gauze strips by braces worn by a patient by preventing direct contact of the braces with the gauze strips.

### CROSS REFERENCE TO THE RELATED APPLICATION

This application claims the benefit of U.S. provisional patent application Ser. No. 63/290,888 filed on Dec. 17, 2021 which is hereby incorporated by reference.

### BACKGROUND OF THE INVENTION

This invention concerns staunching blood flow from a socket left when a wisdom tooth is removed. Such wisdom tooth removals are a common procedure made necessary by a lack of space to accommodate the wisdom teeth.

It is important to stop the bleeding from the socket as soon as possible to accomplish proper healing.

This is currently accomplished by placing one or more strips of sterile medical gauze rolled or folded up over the socket so as to absorb the bleeding from the socket. This use of gauze strips speeds up the process of stopping the bleeding by absorbing the blood and then removing the blood from the socket when the gauze is removed. The gauze is available in rolls three inches in length from which strips of the gauze are cut. It is not uncommon for more than one strip to be used before the bleeding is stopped.

The gauze strips have long been used to absorb blood so as to enable the absorbed blood to be removed from the socket when the gauze strip is removed to promote healing and stop the bleeding.

This ability to absorb and hold blood is believed by the present inventor to be enabled by the relatively large size of the openings in the gauze.

The size of the openings in the gauze avoids any tendency to wick the blood out of the gauze as by capillarity and will be held in the gauze strip to be removed when the gauze strip is removed when the gauze strips or strips have become substantially saturated with blood.

While the gauze strips are effective for absorbing and holding blood, a problem often encountered by patients who have braces in that the braces have a tendency to snag the gauze strip because of the large holes, especially when the patient bites down on the strip as he or she are usually advised to do.

Such snags are difficult to free up, taking much time and requiring the tedious use of tweezers (not shown). This difficulty also effectively delays putting a fresh piece of gauze in place, which can cause a delay in stopping the bleeding.

### SUMMARY OF THE INVENTION

The present inventor has determined that while gauze is an excellent pad material for absorbing blood having large openings due to it being loosely woven, these large openings can easily be seen with the unaided eye compared to the much smaller openings in tightly woven fabrics. However, the large openings in the gauze promote the holding of blood when placed over the socket exposed when a tooth is removed since the tendency of wicking of blood out of the gauze is much less because of the much larger size of the openings in the gauze.

Furthermore, the present inventor believes that the tendency for snagging of the gauze by braces worn by a patient is due to the presence of these large exposed openings in the gauze which are much larger than the openings in a tightly woven fabric as noted above. These openings are believed to enhance gauze's ability to quickly absorb and hold blood and thereby stop the bleeding as mentioned above and thus the use of gauze has long been standard practice. This holding of blood results from an absence of any wicking action causing blood to be expelled out from the gauze. Again, the absence of wicking is due to the much larger holes in gauze. Thus, the blood absorbed by the gauze remains in the gauze and is removed when saturated gauze is removed from the socket.

The present invention provides a blood absorbing pad which will rapidly absorb blood while not having exposed large openings which would create a tendency for the braces to snag the pad.

The pad according to the present invention is comprised of two outer pieces of a tightly woven fabric which is smooth but durable having only tiny openings compared to the large openings in the gauze. The tiny holes in the tightly woven fabric are not large enough to be subject to being snagged by braces.

One or more strips of gauze are enclosed within an enclosure defined by the outer pieces which do not have large openings therein but which rapidly absorbs and transmits blood into the inner gauze layer by a strong wicking action.

The strong wicking action rapidly transmits blood into contact with the inner gauze layer by means of a strong capillary action, so that the inner layer of gauze rapidly absorbs and holds the absorbed blood due to the large openings therein not causing wicking action so as to rapidly absorb and hold the blood while not being subject to snagging of the braces by the presence of the smooth outer layer preventing contact of the braces with the gauze.

The enclosure is formed by first stacking two outer layers (preferably rectangular in shape) of tightly woven fabric which are advantageously sewn or otherwise fixed together along three sides with an open fourth side. The open side allows the enclosure to be manually turned inside out.

A gauze piece is then placed on the outside of one side of the two stacked together fabric pieces and stitched thereto fixedly to one of the fabric pieces.

The sewn together fabric forming the enclosure with the connected gauze strip is then turned inside out to cause the gauze strip to thereby become located within the enclosure defined by the outer fabric pieces to thereby position the gauze layer inside the enclosure defined by the outer fabric pieces. The fourth side of the stacked fabric pieces is then sewn up to block any contact of the gauze with the braces while still allowing the gauze piece to quickly absorb and hold blood wicked into contact with the inner gauze layer. No substantial wicking of blood out of the gauze occurs due to the size of the large openings in the gauze as described above so that when pad is changed absorbed blood is removed from the socket thereby allowing rapid healing.

### DESCRIPTION OF THE DRAWINGS

FIG. 1 is a perspective view of teeth having a prior art folded up gauze layer or layers placed in position over a tooth socket after removal of the tooth, showing braces worn by the patient.

FIG. 2A is an enlarged plan view of a strip of gauze material.

FIG. 2B is a plan view of a greatly enlarged piece of a tightly woven smooth fabric preferably a cotton fabric such as is used on pillow covers to form a smooth surface such as the cotton weave used for quality pillows. At the same time the cotton fabric pieces causes the blood to quickly pass through the same due to the strong wicking action caused by the small size of the holes therein as will be understood by those skilled in the field.

FIG. 3 is a perspective view of two of the fabric pieces stacked atop each other which are sewn together on three sides thereof, with the fourth side left open, and a gauze layer sewn to the top of one of the two fabric pieces stacked together.

FIG. 4 is a top view of the stacked gauze and fabric pieces shown in FIG. 3.

FIG. 5 is a plan view of the assembly of FIGS. 3 and 4 which has been turned inside out to enclose the gauze strip, with the fourth side then sewn shut.

### DETAILED DESCRIPTION

In the following detailed description, certain specific terminology will be employed for the sake of clarity and a particular embodiment described in accordance with the requirements of 35 USC 112, but it is to be understood that the same is not intended to be limiting and should not be so construed inasmuch as the invention is capable of taking many forms and variations within the scope of the appended claims.

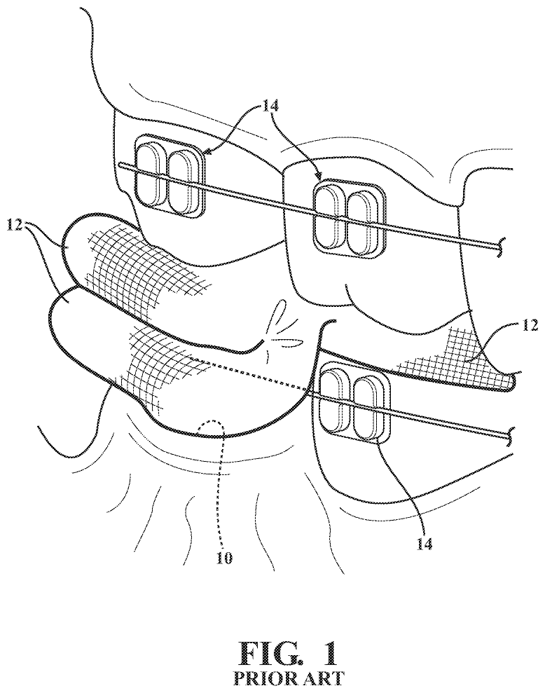

Referring to the drawings, FIG. 1 shows a socket 10 in a person's gum which is exposed after a tooth has been removed.

Strips of gauze 12 usually folded or rolled up are placed over the socket 10 to absorb bleeding which occurs after the tooth is removed. Braces 14 are worn by many dental patients which often sink into the gauze 12 and become snagged therein as described above.

As described above, the gauze 12 has relatively large openings 18 formed therein and the presence of such large openings in the gauze often cause a snagging of the braces 14 when the teeth push into the gauze 12.

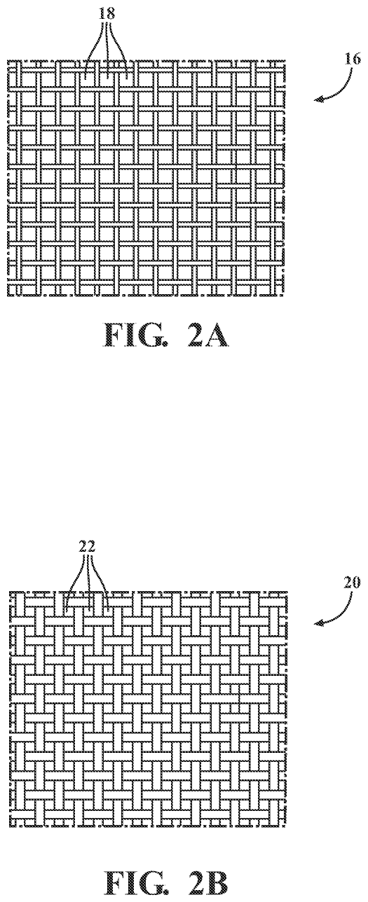

According to the present invention such snagging is avoided by enclosing the gauze 12 in an enclosure defined by tightly woven fabric pieces 20 which will not be penetrated by the braces since only tiny holes are formed therein. FIGS. 2A and 2B show the approximate great difference in size of the openings 18 and 22.

The gauze 16 is loosely woven such that much larger openings 18 result (FIG. 2A).

Since tightly woven fabric pieces 20 such as cotton fabrics have much smaller openings 22, these tiny openings 22 resist penetration and snagging of the braces 14 to a much greater extent.

Cotton fabric 20 as used in quality pillows has been found to have much greater resistance to penetration by the braces 14 as well as presenting a smoother surface so as to resist being snagged by the braces 14.

According to the concept of the invention, those characteristics are taken advantage of by enclosing the gauze 12 in an enclosure defined by the tightly woven fabric pieces 20 to resist snagging.

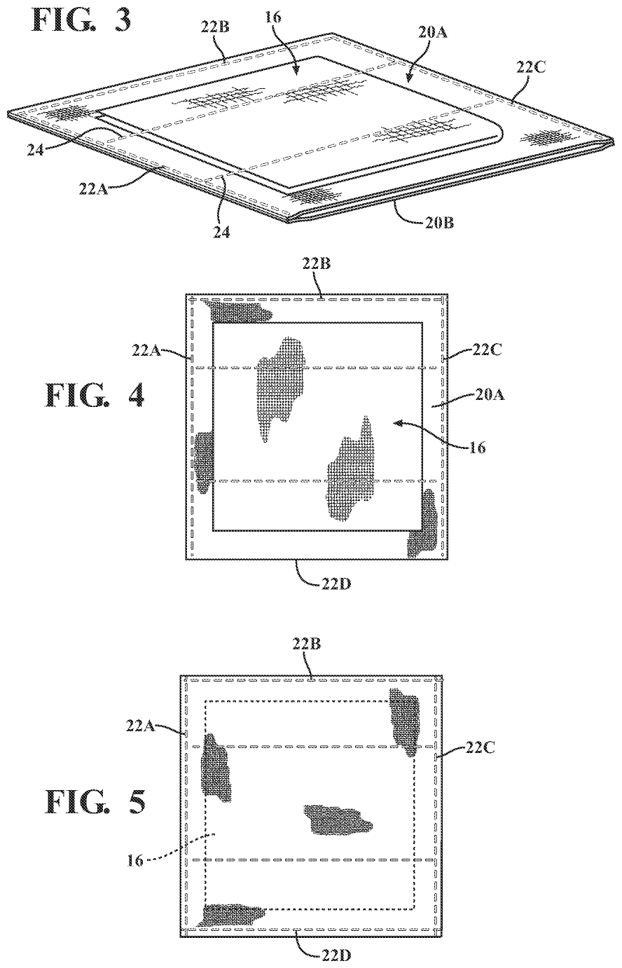

FIG. 3 shows one or more strips of gauze 12 are which located atop a pair of preferably rectangular sheets 20A, 20B of tightly woven fabric pieces 20.

Three sides 22A, 22B, 22C thereof are connected together as by being sewn by a sewing machine while the fourth side is temporarily left open.

A layer of gauze 12 is located centered on one of the fabric sheets 20A or 20B, and fixed in that location as by hand sewn seams 24 extending across one of the fabric pieces 20A or 20B and the gauze strip or strips 16.

The sheet pieces 20A and 22B are then turned inside out so that the gauze strip or strips 16 are now enclosed within the space between the fabric pieces 20A, 20B.

The fourth side is then sewn shut (FIG. 5) so that the gauze strip or strips 16 are completely enclosed by the tightly woven fabric pieces 20A, 20B, so that the braces 14 cannot contact the gauze strip or strips 16.

It is believed that capillarity causes a large volume of blood to be forced through the tiny openings 22 in the fabric pieces 20A, 20B by a wicking action so as to rapidly flood the gauze 16 with blood despite the tiny size of the openings 22.

This has been found to eliminate or greatly reduce the snagging problem described.

1. A pad able to absorb blood flowing out of a patient's socket exposed in his or her gums when a tooth is removed by being placed in position over the socket, said pad comprising an enclosure defined by two tightly woven stacked fabric pieces, sewn together, with a layer of loosely woven gauze material held within said enclosure so as to prevent any direct contact between said gauze and braces worn by a patient so that said braces are prevented from snagging said gauze material.

2. The pad according to claim 1 where said enclosure defining pieces are stacked together to have a perimeter by stacked fabric pieces connected together around a perimeter thereof with said gauze material disposed lying between and within the perimeter each of said fabric pieces.

3. The pad according to claim 1 wherein said tightly woven fabric pieces are sewn together along perimeters thereof.

4. The pad according to claim 3 wherein said tightly woven fabric and gauze pieces are each substantially rectangular.

5. The pad according to claim 3 wherein said gauze strip is on the order of 2 inches wide and long.

6. A method of absorbing blood from a bleeding socket exposed by removal of a tooth comprising the steps of disposing one or more strips of gauze in an enclosure defined by pieces of tightly woven fabric to form a pad and placing said pad over said socket so that blood is rapidly passed through said fabric pieces into said gauze strip by a wicking action.

7. A method for forming a blood absorbent pad for use in dentistry comprising the steps of enclosing one or more gauze strips having large openings in an enclosure defined by two tightly woven fabric pieces each having much smaller openings therein than in said one or more gauze strips whereby blood is forced through said fabric pieces by a wicking action into said one or more gauze strips where it is retained and removed when said one or more gauze strips are removed.

8. The method of forming said pad according to claim 7 further including the step of sewing said two fabric pieces together along three sides thereof leaving a fourth side thereof temporarily open;
sewing a gauze strip to one of said pieces and thereafter turning said two fabric pieces inside out so that said gauze strip is enclosed between said fabric pieces, and then sewing the fourth side to be closed.
